(19) United States
(12) Patent Application Publication
(10) Pub. No.: US 2019/0049464 A1
(43) Pub. Date: Feb. 14, 2019

(54) BLOOD BIOMARKER FOR DETECTING ARTERIOSCLEROSIS

(71) Applicants: Public University Corporation Yokohama City University, Yokohama-shi, Kanagawa (JP); TOSOH CORPORATION, Shunan-shi, Yamaguchi (JP)

(72) Inventors: Utako YOKOYAMA, Yokohama-shi (JP); Yoshihiro ISHIKAWA, Yokohama-shi (JP); Noriaki ARAKAWA, Yokohama-shi (JP); Munetaka MASUDA, Yokohama-shi (JP); Tomoaki ISHIGAMI, Yokohama-shi (JP); Shin-ichi SUZUKI, Yokohama-shi (JP); Norihisa OHTAKE, Ayase-shi (JP); Hiroki KOBORI, Ayase-shi (JP)

(73) Assignees: Public University Corporation Yokohama City University, Yokohama-shi, Kanagawa (JP); TOSOH CORPORATION, Shunan-shi, Yamaguchi (JP)

(21) Appl. No.: 16/077,704

(22) PCT Filed: Feb. 15, 2017
(86) PCT No.: PCT/JP2017/005565

§ 371 Date: Aug. 13, 2018

(30) Foreign Application Priority Data
Feb. 16, 2016 (JP) 2016-026494

(51) Int. Cl. G01N 33/68

(52) U.S. Cl. CPC G01N 33/6887; G01N 2800/323

(57) ABSTRACT

Novel means capable of detecting an arteriosclerotic lesion in a body by a less invasive, simple method is disclosed. The method for detecting arteriosclerosis according to the present invention comprises measuring the myosin heavy chain 11 level in a blood sample isolated from a subject. A myosin heavy chain 11 level higher than that of a healthy individual is indicative of the presence of arteriosclerosis in the subject. Since the present invention enables detection of arteriosclerosis by a blood test, a less invasive, simple test can be carried out. Unlike methods for testing only particular regions such as carotid artery ultrasonography, the present invention is expected to reflect the conditions of blood vessels in the whole body.

### TECHNICAL FIELD

The present invention relates to a method for detecting arteriosclerosis using as an indicator the blood myosin heavy chain 11 level.

### BACKGROUND ART

Due to aging of the population and changes in the lifestyle, diseases mainly involving arteriosclerosis, such as coronary artery diseases, peripheral arterial diseases, and aortic aneurysm, have been increasing. In order to improve prognosis in arteriosclerotic diseases, it is necessary to diagnose arteriosclerotic diseases by a non-invasive, relatively simple test and design an appropriate examination and treatment plan.

Examples of the method for non-invasively detecting arteriosclerosis include methods in which the intima media thickness, the presence of plaques, and the like are measured by carotid artery ultrasonography. However, in such methods, diagnosis is made based on observation of the limited region, carotid artery only, and therefore the methods are not necessarily capable of detecting arteriosclerosis in other regions.

Known risk factors for development of arteriosclerosis include diabetes, smoking, hyperlipidemia, and hypertension (Non-Patent Document 1). However, at present, thanks to the progress of pharmacotherapy for hypertension and hyperlipidemia, the risk of arteriosclerosis cannot be judged based solely on these classical risk factors. Since arteriosclerosis itself is asymptomatic, a patient cannot become aware of its progression. Thus, biomarkers capable of diagnosis of arteriosclerosis have been demanded.

In recent years, an increased number of studies have been carried out for biomarkers based on the mechanism of development of arteriosclerosis. Progression of arteriosclerosis is known to involve disorders of vascular endothelial functions, abnormality of the coagulation-fibrinolytic system, inflammation, lipid metabolism abnormality, and the like, and association between proteins related to these factors and acute coronary syndrome has been shown (Non-Patent Document 2). However, since increases in these proteins may also occur due to infections, cancers, traumas, and the like, they are poorly specific to blood vessels, so that arteriosclerosis cannot be diagnosed only by a single protein molecule. In Non-Patent Document 2, not less than 20 factors associated with lipids, metabolism, coagulation, angiogenesis, renal function/inflammation, oxidative stress, and the like have been studied. However, no biomarker better than the classical risk factors has been discovered.

In Patent Document 1, a large number of proteins including tumor necrosis factor-α precursor have been proposed as biomarkers capable of predicting cardiovascular events. The cardiovascular events include various arteriosclerotic diseases such as aortic aneurysm rupture, myocardial infarction, stroke, and peripheral arterial diseases. However, these biomarkers do not diagnose arteriosclerosis itself.

Patent Document 2 discloses blood YKL-40 as a prognostic biomarker for heart diseases caused by atherosclerosis. This biomarker also does not diagnose arteriosclerosis itself. Further, since YKL-40 is found in blood also in cases of cancer and inflammation (Non-Patent Documents 3 and 4), its disease specificity is low.

Patent Document 3 discloses a method for evaluating an arteriosclerotic lesion using as indicators marker proteins whose expression changes along with the progression of the arteriosclerotic lesion. However, since these marker proteins have been identified using arteriosclerosis model animals, whether or not they are capable of evaluation of human arteriosclerotic lesions is unclear. Further, since the method is based on investigation of protein expression levels in the arterial tissue lesion, the method, similarly to carotid artery ultrasonography, is not necessarily capable of detecting the presence of arteriosclerosis in regions other than the region to be examined.

### PRIOR ART DOCUMENT(S)

### Patent Document(s)

Patent Document 1: JP 2010-534852 A
Patent Document 2: JP 2011-510308 A
Patent Document 3: WO 2012/067180

### Non-Patent Document(s)

Non-Patent Document 1: Anderson K M. et al., Am Heart J, 1991; 121: 293-8
Non-Patent Document 2: Blankengerg S. et al., Circulation, 2010; 121: 2388-97
Non-Patent Document 3: Kring T S., et al., Biomark Cancer, 2015; 7: 57-61
Non-Patent Document 4: Ural U M., et al., Clin Exp Obstet Gynecol, 2015; 42: 495-7

### SUMMARY OF THE INVENTION

### Problems to be Solved by the Invention

An object of the present invention is to provide novel means capable of detecting an arteriosclerotic lesion in a body by a less invasive, simple method.

### Means for Solving the Problems

As a result of intensive study, the present inventors discovered that the blood myosin heavy chain 11 level in arterial disease patients having a severe arteriosclerotic lesion is much higher than that in healthy individuals, thereby completing the present invention.

That is, the present invention provides a method for detecting arteriosclerosis, comprising measuring the myosin heavy chain 11 level in a blood sample isolated from a subject, wherein a myosin heavy chain 11 level higher than that in a healthy individual is indicative of the presence of arteriosclerosis in the subject. The present invention also provides an arteriosclerosis detection kit comprising an anti-myosin heavy chain 11 antibody or an antigen-binding fragment thereof.

### Effect of the Invention

The present invention revealed, for the first time, that myosin heavy chain 11 can be used as a blood biomarker reflecting the presence of arteriosclerosis in a body. Since this allows detection of arteriosclerosis by a blood test, a less invasive, simple test can be carried out. Further, since the method, unlike carotid artery ultrasonography, is not based on examination of a particular region only, it is expected to reflect the conditions of blood vessels in the whole body. By detecting arteriosclerosis itself by the present invention, the risk of development of an arteriosclerotic disease can be known before the development. This can provide a chance for promoting self-improvement of the lifestyle, leading to prevention of development of the disease and contributing to reduction of the medical cost.

Moreover, in a patient at an early stage of development of an arteriosclerotic disease wherein the patient hardly has subjective symptoms, knowing of the presence of severe arteriosclerosis by the method of the present invention may lead to early detection of the disease, which enables design of a treatment plan at an earlier phase, contributing to improvement of the life prognosis of the patient. By the present invention, data useful for detection of the presence of an arteriosclerotic lesion can be provided, so that evaluation and diagnosis of the arteriosclerosis itself by a physician can be greatly assisted.

### BRIEF DESCRIPTION OF THE DRAWINGS

FIG. 1-1 shows the result of comparison of the measured value of the blood myosin heavy chain 11 level between a normal group and an arteriosclerosis patient group. A significant difference between the groups was found at P<0.0001 (Welch's t-test).

FIG. 1-2 shows the result of evaluation, by ROC analysis, of the capacity of the blood myosin heavy chain 11 level to detect the arteriosclerosis patient group.

FIG. 2-1 shows the result of comparison of the measured value of the blood myosin heavy chain 11 level among the following three groups: a normal group, an arteriosclerosis patient group that received PCI, and an arteriosclerosis patient group that received EVT. *** indicates a significant difference from the normal group at P<0.0001 (Welch's t-test).

FIG. 2-2 shows the result of evaluation, by ROC analysis, of the capacity of the blood myosin heavy chain 11 level to detect each arteriosclerosis patient group.

FIG. 3-1 shows the result of comparison of the measured value of the blood myosin heavy chain 11 level among the following three groups: a Japanese normal group, an arteriosclerosis patient group that received PCI, and an arteriosclerosis patient group that received EVT. *** indicates a significant difference from the normal group at P<0.0001 (Welch's t-test).

FIG. 3-2 shows the result of evaluation, by ROC analysis, of the capacity of the blood myosin heavy chain 11 level to detect each arteriosclerosis patient group.

FIG. 4 shows the result of investigation of correlation between the blood MYH11 level and the blood CRP level in 46 arteriosclerosis patients.

FIG. 5 shows the result of evaluation, by ROC analysis, of the capacity of the blood CRP level to detect each arteriosclerosis patient group.

FIG. 6 shows the result of investigation of correlation between the blood MYH11 level and the Brinkman index in Japanese healthy individuals, PCI patients, and EVT patients.

FIG. 7 shows the result of measurement of the molar concentrations of two target peptides (ALELDPNLYR and LEVNMQALK) of MYH11 present in a sample using internal standard peptides as indices.

FIG. 8 shows the result of comparison of the blood MYH11 level before the intervention, immediately after the intervention, or within several days after the intervention between a surviving group and a death group (observation period, 1 to 2.5 years) of arteriosclerosis patients who received PCI or EVT.

FIG. 9-1 shows alignment of the amino acid sequences of four isoforms of myosin heavy chain 11.

FIG. 9-2 shows alignment of the amino acid sequences of four isoforms of myosin heavy chain 11 (continued from FIG. 9-1).

FIG. 9-3 shows alignment of the amino acid sequences of four isoforms of myosin heavy chain 11 (continued from FIG. 9-2).

FIG. 9-4 shows alignment of the amino acid sequences of four isoforms of myosin heavy chain 11 (continued from FIG. 9-3).

FIG. 9-5 shows alignment of the amino acid sequences of four isoforms of myosin heavy chain 11 (continued from FIG. 9-4).

### MODE FOR CARRYING OUT THE INVENTION

For myosin heavy chain, isoforms such as 5, 7, 10, and 11 are known. Among these, myosin heavy chain 11 (Gene ID 4629) is the target of the present invention. Myosin heavy chain 11 is an isoform specifically expressed in smooth muscle, and also called smooth muscle myosin heavy chain. Two myosin heavy chain 11 molecules expressed in smooth muscle are combined with two pairs of myosin light chain molecules, each pair consisting of the same type of the light chain, to form a hexameric smooth muscle myosin molecule.

It is known that myosin heavy chain 11 has four isoforms, SM1A, SM1B, SM2A, and SM2B (GenBank Accession Nos. NP_002465.1, NP_001035203.1, NP_074035.1, and NP_001035202.1, whose amino acid sequences are shown in SEQ ID NOs:1 to 4, respectively), which isoforms vary mainly in the C-terminal sequence. The isoform of myosin heavy chain 11 to be measured in the present invention is not limited. All four isoforms may be measured, or only part of the isoforms may be measured.

The blood myosin heavy chain 11 level can be investigated by measuring myosin heavy chain 11 protein or a fragment thereof in a blood sample. Since myosin heavy chain 11 is not a secretory protein, it is thought to be present also in a fragmented form in blood. The myosin heavy chain 11 protein or the fragment thereof to be measured includes one present in blood as a monomer and one present in blood in a state where it is bound to or associated with another protein or the like (for example, one present in blood in a state where it constitutes a complete or partially-degraded smooth muscle myosin molecule). Thus, the term “myosin heavy chain 11” used to refer to a blood biomarker for detection of arteriosclerotic lesion includes not only the full-length myosin heavy chain 11 protein present in blood in a monomeric state and a partial fragment thereof, but also myosin heavy chain 11 protein and a partial fragment thereof in a state where they are bound to or associated with (an)other protein(s) or protein fragment(s).

The blood sample may be serum or plasma. Plasma may be preferably used.

The arteriosclerosis to be detected in the present invention includes arteriosclerosis at any stage between early stage and advanced, late stage. Arteriosclerosis can be pathologically classified into the following three types: atherosclerosis, arteriolosclerosis, and medial calcification. The arteriosclerosis to be detected in the present invention includes these three types. A typical example of the arteriosclerosis to be detected is atherosclerosis.

Examples of diseases caused by arteriosclerosis (arteriosclerotic diseases) include ischemic heart diseases (angina pectoris, myocardial infarction), aortic aneurysm (thoracic/abdominal aortic aneurysm, iliac aneurysm, and the like), arteriosclerosis obliterans, aortic stenosis, and cerebrovascular diseases (cerebral infarction, stroke). By detecting arteriosclerosis itself by the present invention, the risk of development of such diseases can be known before their development. Thus, self-improvement of the lifestyle can be promoted, which leads to prevention of the development of the arteriosclerotic diseases. For subjects in whom severe arteriosclerosis is detected, early detection and early treatment of the diseases become possible by carrying out detailed examination and subsequent careful follow-up.

The means for measuring the blood myosin heavy chain 11 level is not limited as long as it is capable of measuring a polypeptide. Examples of the method for measuring a polypeptide include immunoassays and mass spectrometry. Immunoassays may be preferably used for the measurement of the myosin heavy chain 11 level in the present invention since they do not require a large-scale apparatus and can be carried out by a simple measurement operation. Antibodies capable of detecting myosin heavy chain 11 are known, and there are commercially available products of such antibodies. A commercially available anti-myosin heavy chain 11 antibody is typically a non-human animal polyclonal or monoclonal antibody prepared with a non-human animal such as mouse or rabbit. Since amino acid sequences of myosin heavy chain 11 and base sequences encoding them are known as described above, an anti-myosin heavy chain 11 antibody that specifically recognizes myosin heavy chain 11 may be prepared by a conventional hybridoma method or the like, and may then be used.

The anti-myosin heavy chain 11 antibody may be a polyclonal antibody or a monoclonal antibody. An antiserum may be used as a polyclonal antibody. These antibodies may be prepared by well-known conventional methods.

More specifically, an anti-myosin heavy chain 11 polyclonal antibody can be obtained by, for example: immunizing a non-human animal with a myosin heavy chain 11 protein or partial fragment thereof prepared by chemical synthesis or genetic engineering, together with an adjuvant when appropriate; obtaining an antiserum from blood collected from the immunized animal; and then purifying the polyclonal antibody (non-human animal anti-myosin heavy chain 11 polyclonal antibody) from the antiserum. Usually, in order to increase the antibody titer in the immunized animal, the immunization is carried out multiple times over a period of several weeks. The antibody in the antiserum can be purified by, for example, fractionation by ammonium sulfate precipitation, fractionation by anion chromatography, or affinity column purification.

An example of well-known methods for preparation of a monoclonal antibody is the hybridoma method. More specifically, a non-human animal anti-myosin heavy chain 11 monoclonal antibody can be obtained by, for example, collecting antibody-producing cells such as spleen cells or lymphocytes from a non-human animal immunized as described above; fusing these cells with myeloma cells to prepare hybridomas; selecting a hybridoma producing an antibody that binds to myosin heavy chain 11 protein; growing the selected hybridoma; and then obtaining the non-human animal anti-myosin heavy chain 11 monoclonal antibody from the culture supernatant.

The immunoassays themselves are well known in the art. Immunoassay methods can be classified based on the reaction mode into the sandwich method, competition method, agglutination method, Western blotting method and the like, or can be classified based on the label into the enzyme immunoassay, radioimmunoassay, fluorescence immunoassay and the like. In the present invention, any immunoassay method capable of quantitative detection may be used. Preferred Examples of such a method include, but are not limited to, sandwich methods such as sandwich ELISA.

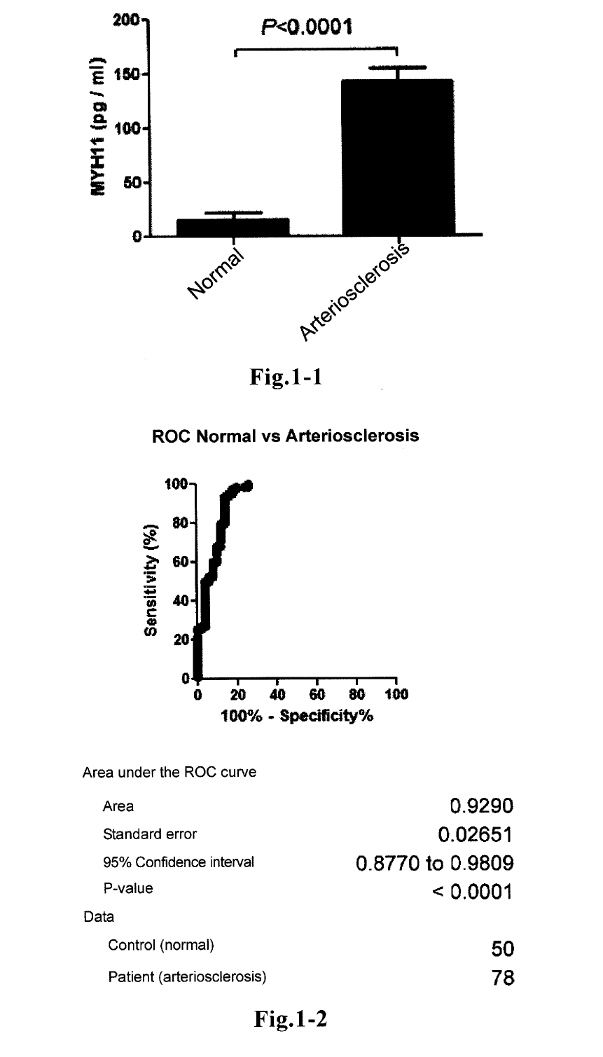

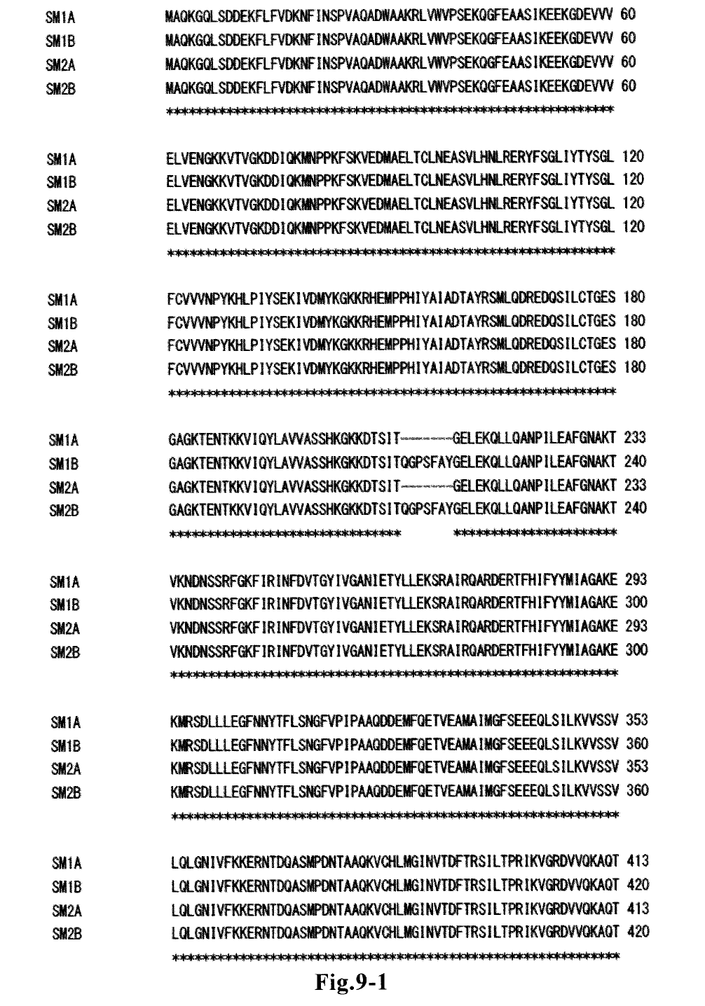

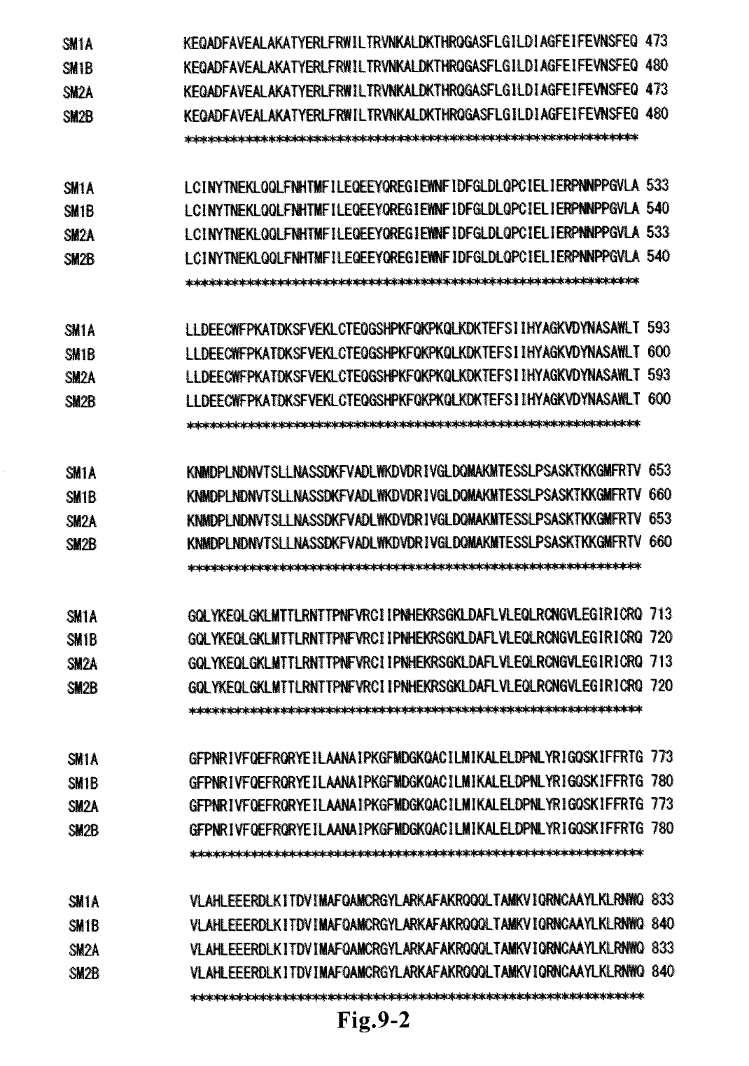

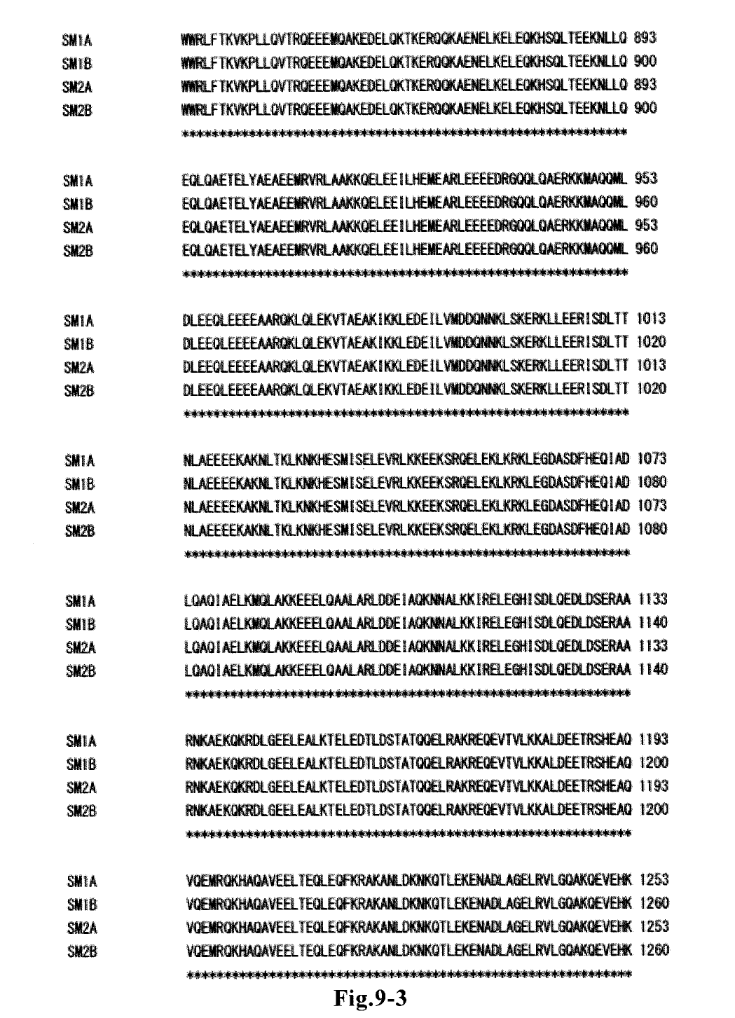

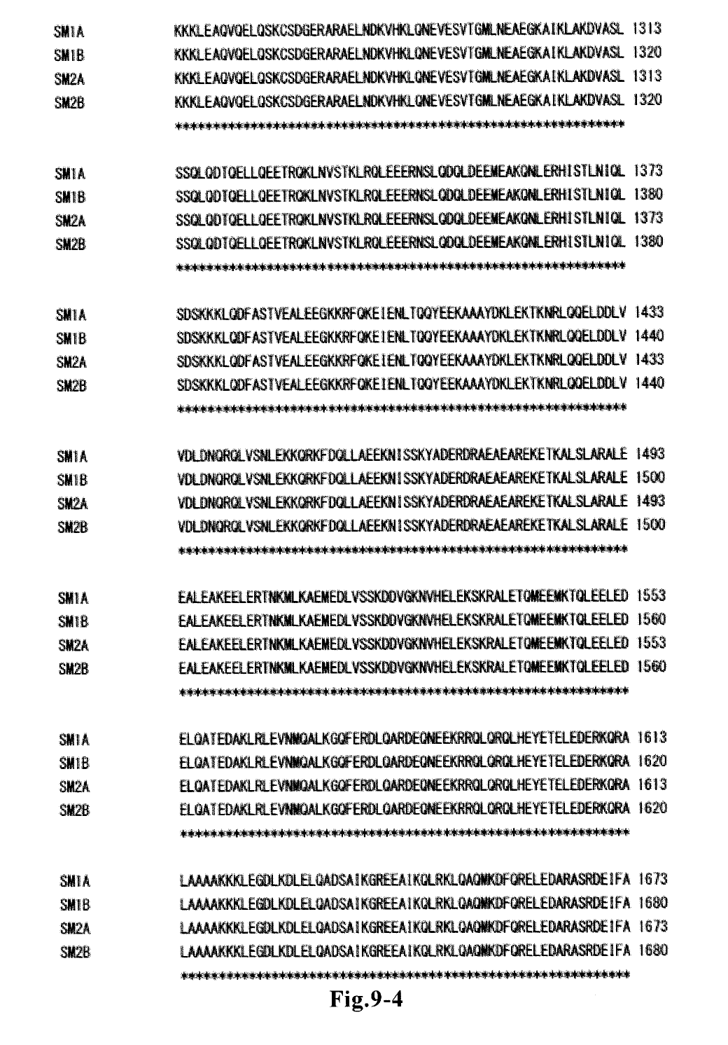

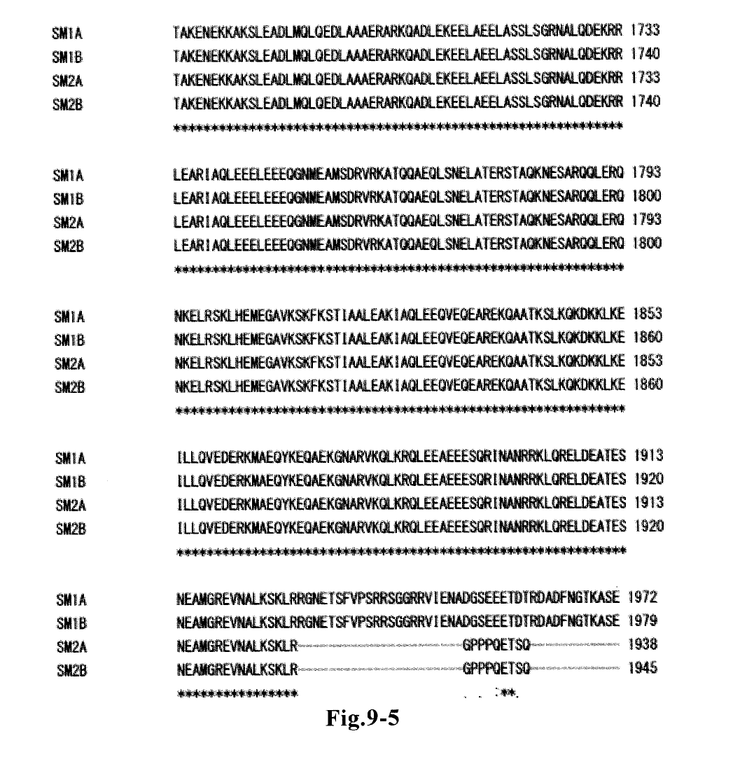

As described above, two myosin heavy chain 11 molecules are contained in a smooth muscle myosin molecule. Such a complex containing two myosin heavy chain 11 protein molecules or two fragments thereof can be measured by a sandwich method using the same antibodies that recognize the same site of myosin heavy chain 11. When a monomeric myosin heavy chain 11 protein or a fragment thereof, or a complex containing just one myosin heavy chain 11 molecule is to be measured by a sandwich method, two kinds of antibodies that recognize different sites of myosin heavy chain 11 may be used. When a polyclonal antibody is employed, the same polyclonal antibody can be used both as a labeled antibody and as a solid-phase antibody to measure the protein including the monomeric molecule and its fragments. As described above, four isoforms of myosin heavy chain 11 are known. All four isoforms can be comprehensively measured by using an antibody that recognizes a region commonly shared by the four isoforms. By using antibodies specific to the four isoforms, respectively, the isoforms can be separately measured, or a particular isoform(s) can be measured. As shown in FIGS. 9-1 to 9-5, the regions of amino acid positions 1 to 211 and 219 to 1936 of SEQ ID NO:4 are regions that are commonly shared by the four isoforms shown in SEQ ID NOs:1 to 4. By using these regions or a part of these regions as an immunogen in preparation of the antibody, an antibody reactive to all four isoforms can be prepared. The region of amino acid positions 212 to 219 of SEQ ID NO:4 is a region specific to SM1B and SM2B; the region of amino acid positions 1937 to 1945 of SEQ ID NO:4 is a region specific to SM2A and SM2B; and the region of amino acid positions 1930 to 1972 of SEQ ID NO:1 is a region specific to SM1A and SM1B. Accordingly, by appropriate combination of antibodies that recognize these regions or partial regions thereof, the isoforms can be separately measured, or a particular isoform(s) can be measured.

In the present invention, an antibody that recognizes the C-terminal region of myosin heavy chain 11 protein may be typically preferably used. However, an antibody that recognizes the N-terminal region may be used, or an antibody that recognizes the C-terminal region and an antibody that recognizes the N-terminal region may be used in combination. In the present invention, the N-terminal region means a region in the N-terminal side including the head portion, which is constituted by the motor domain and the regulatory domain, and part of the tail portion having a coiled-coil structure; and the C-terminal region means a region in the C-terminal side including a major part of the tail portion having a coiled-coil structure. More specifically, for example, in the amino acid sequence of SEQ ID NO:4, a region from around the amino acid position 840 to the N-terminus is the N-terminal region, and a region from around the amino acid position 840 to the C-terminus is the C-terminal region. The antibody that recognizes the C-terminal region means an antibody which has its epitope in the C-terminal region of myosin heavy chain 11 protein and binds to a site in the C-terminal region. The term “antibody that recognizes the N-terminal region” can be defined in the same manner.

The immunoassays themselves are well known, and do not need to be explained in the present description. Briefly, in a sandwich immunoassay, for example, an antibody that binds to myosin heavy chain 11 is immobilized on a solid phase (immobilized antibody), and then reacted with a sample. After washing, the solid phase is reacted with a labeled antibody prepared by adding a label to an antibody that binds to myosin heavy chain 11 at a site which is the same as or different from the site to which the immobilized antibody binds.

After washing, the labeled antibody bound to the solid phase is measured. Each of the immobilized antibody and the labeled antibody may be either a polyclonal antibody or a monoclonal antibody. In the immunoassay, an antigen-binding fragment(s) of the antibody(ies) may be used instead of the antibody(ies).

The “antigen-binding fragment” means an antibody fragment which maintains the binding capacity to the corresponding antigen (antigen-antibody reactivity) that the original antibody has, such as the Fab fragment or F(ab′)₂ fragment of immunoglobulin. It is well known that such antigen-binding fragments can also be used in immunoassays, and the fragments are useful similarly to their original antibodies. Fab fragments and F(ab′)₂ fragments can be obtained, as is well known, by treating antibodies with a protease such as papain or pepsin. The antigen-binding fragment is not limited to Fab fragments and F(ab′), fragments, and may be any fragment maintaining the binding capacity to the corresponding antigen. It may also be one prepared by a genetic engineering method. An antibody prepared by expressing its single chain fragment of variable region (scFv) in E. coli by a genetic engineering method may also be used. The method for preparing scFv is also well known. scFv can be prepared by: extracting mRNA from a hybridoma prepared as described above; preparing single-stranded cDNA therefrom; performing PCR using primers specific to the II-chain and L-chain of immunoglobulin to amplify the immunoglobulin H-chain gene and L-chain gene; linking these genes together via a linker; introducing the resultant to a plasmid vector after adding appropriate restriction sites thereto; transforming E. coli with the resulting plasmid vector; and then recovering scFv from the transformed E. coli. Such an scFv is also included in the “antigen-binding fragment”.

Measurement of the labeled antibody can be carried out by measuring a signal from the labeling substance. The method for measuring the signal is appropriately selected depending on the type of the labeling substance. For example, when an enzyme label is employed, the substrate of the enzyme may be added into the reaction system, and the amount of coloring or luminescence caused by the enzyme reaction may be measured using an absorptiometer or a luminometer. Myosin heavy chain 11 in a sample can be quantified by beforehand performing an immunoassay of standard samples containing myosin heavy chain 11 at various known concentrations using an anti-myosin heavy chain 11 antibody or an antigen-binding fragment thereof, plotting the correlation between the amount of signal from the label and the myosin heavy chain 11 concentration in each standard sample to prepare a calibration curve, carrying out the same operation for a sample whose myosin heavy chain 11 concentration is unknown to measure the amount of signal from the label, and then applying the measured value to the calibration curve.

In the case where both of the immobilized antibody and the labeled antibody are monoclonal antibodies, whether the combination of the monoclonal antibodies is desirable or not can be easily determined by actually performing the immunoassay. If desired, whether the antibodies recognize different epitopes or not may be determined by identifying the site recognized by each antibody. The identification of the site recognized by each antibody can be carried out by a conventional method well known in the art. Briefly, for example, myosin heavy chain 11, which is the corresponding antigen, is partially digested with a protease such as trypsin. A solution of the partial digestion product is then passed through an affinity column to which the antibody whose site of recognition is to be determined is bound, thereby allowing the digestion product to be bound to the column. Thereafter, the bound digestion product is eluted and subjected to conventional mass spectrometry to identify the site recognized by the antibody.

A blood myosin heavy chain 11 level higher than that of a healthy individual is indicative of the presence of arteriosclerosis. By measurement of the blood myosin heavy chain 11 level, data helpful for detection of arteriosclerosis can be provided. The term “detection of arteriosclerosis” includes detection of severe arteriosclerosis. When the measured value of the blood myosin heavy chain 11 level in a subject is higher than that in a healthy individual, it can be judged that the subject may have an arteriosclerotic lesion. Furthermore, the higher the blood myosin heavy chain 11 level, the higher the possibility of the presence of an arteriosclerotic lesion in the body of the subject can be judged to be, or the higher the severity of the arteriosclerosis can be judged to be. The terms “arteriosclerosis with high severity” and “severe arteriosclerosis” include a state where the number of arteriosclerotic lesions present in the body is large, and a state where the degree of progression of arteriosclerotic lesions present in the body is high. The terms “arteriosclerosis with a high degree of progression” and “advanced arteriosclerosis” mean arteriosclerosis at a level at which a stenosis of not less than 50%, for example, not less than 60%, not less than 70%, or not less than 75% is found by angiography. The rate of stenosis as observed by angiography represents, as is well known in the art, to what extent the blood vessel is narrowed as compared to the diameters of the blood vessel at sites upstream and downstream of the stenosis, which sites are thought to be in a healthy state. For example, 75% stenosis means that the diameter of the imaged site is 25% relative to the blood vessel diameters at sites upstream and downstream of the imaged site, which are taken as 100%.

Whether or not the blood myosin heavy chain 11 level measured is higher than that in a healthy individual can be judged by, for example, employing a healthy reference value determined from blood myosin heavy chain 11 levels measured in a large number of healthy individuals as a cut-off value, and comparing the measured level with the cut-off value. Healthy individuals are individuals who are free of arteriosclerosis, and individuals having symptoms of arteriosclerotic diseases are of course excluded from the healthy individuals. The healthy individuals may be selected based on self-declaration by individuals free of arteriosclerosis. Or, for example, individuals free of lifestyle-related diseases known to be risk factors for arteriosclerosis, such as hyperlipidemia, hypertension, diabetes, and obesity, as well as free of arteriosclerotic diseases may be selected. In the selection of healthy individuals, the absence of the smoking habit, the family history of arteriosclerotic diseases, etc. may also be further taken into account. The healthy reference value may be, for example, the average of blood myosin heavy chain 11 levels in healthy individuals, or the value obtained by adding the standard error to the average. When the measured value of the blood myosin heavy chain 11 level in a subject is higher than the cut-off value, there is a possibility that the subject has an arteriosclerotic lesion. The higher the measured value, the higher the possibility of the presence of the arteriosclerotic lesion can be judged to be, or the higher the severity of the arteriosclerosis can be judged to be. The cut-off value may be set according to age (for example, cut-off values for subjects under 30 years old, in the 30s, in the 40s, in the 50s, in the 60s, and in the 70s or older may be set, respectively).

The present invention can also be carried out for the purpose of detecting especially severe arteriosclerosis that has become advanced to a level at which therapeutic treatment is required. In such a case, a severe arteriosclerosis reference value may be determined based on blood myosin heavy chain 11 levels measured in a group of patients who have developed a severe arteriosclerotic disease such as a coronary artery disease or a peripheral arterial disease. As a severe arteriosclerosis reference value, the average of measured values of the blood myosin heavy chain 11 level in the said group of patients, the value obtained by subtracting the standard error from the average, or the like may be employed. When the measured value of a subject is close to or exceeds the severe arteriosclerosis reference value, it can be judged that the subject may have especially severe arteriosclerosis. By carrying out a detailed examination for such a subject as soon as possible, and carrying out an immediate therapeutic treatment if abnormality is found, or carefully following up, early detection and early treatment of arteriosclerotic disease become possible.

The present invention can also be used for judgment of a therapeutic effect of a therapeutic treatment for arteriosclerosis or an arteriosclerotic disease, or for judgment of a therapeutic effect of therapeutic candidates in a clinical test or the like. When a patient who received a therapeutic treatment shows a decreased blood myosin heavy chain 11 level as compared to that before the therapeutic treatment, it can be judged that the therapeutic treatment has produced a therapeutic effect. When a patient who received administration of a therapeutic candidate shows a decreased blood myosin heavy chain 11 level as compared to that before the administration, it can be judged that the therapeutic candidate has a therapeutic effect on arteriosclerosis or an arteriosclerotic disease.

An anti-myosin heavy chain 11 antibody or an antigen-binding fragment thereof can be provided as an arteriosclerosis detection kit. As described above, the anti-myosin heavy chain 11 antibody may be an antibody that recognizes a region commonly shared by the four isoforms and can comprehensively measure all the four isoforms, or may be an isoform-specific antibody/antibodies that separately recognize(s) each of the four isoforms. If the anti-myosin heavy chain 11 antibody is an isoform-specific antibody/antibodies, the arteriosclerosis detection kit may comprise all antibodies specific to each of the four isoforms, or may comprise part of such antibodies. A preferred example of the kit is a kit comprising an antibody that recognizes the C-terminal region of myosin heavy chain 11 protein. The kit may also be a kit comprising an antibody that recognizes the N-terminal region, or a kit comprising both an antibody that recognizes the C-terminal region and an antibody that recognizes the N-terminal region.

The arteriosclerosis detection kit usually comprises instructions for use. In cases of a detection kit based on a sandwich method, the kit may also comprise a plate or particles such as magnetic particles as a solid phase. The anti-myosin heavy chain 11 antibody or the antigen-binding fragment thereof may be contained in the kit as a labeled antibody and an immobilized antibody. The labeled antibody and the immobilized antibody may be the same anti-myosin heavy chain 11 antibody or antigen-binding fragment thereof, or may be different anti-myosin heavy chain 11 antibodies or antigen-binding fragments thereof.

### Examples

The present invention is described below in more detail by way of Examples. However, the present invention is not limited to the Examples below.

### 1. Quantification of Blood Myosin Heavy Chain 11 by Sandwich ELISA (Part 1)

### <Materials and Methods>

The quantification was carried out for patients who developed an arterial disease such as stenosis, infarction, or aneurysm in a coronary artery, aorta, or peripheral blood vessel due to progression of arteriosclerosis and received catheterization or stenting. The presence of an arteriosclerotic lesion in each patient was confirmed by angiography (confirmation of stenosis or infarction) or CT (confirmation of calcification). Patients whose arteriosclerosis could not be confirmed were excluded. Plasma was separated from blood collected from each patient before the intervention, immediately after the intervention, or within several days after the intervention, and the myosin heavy chain 11 (MYH11) level in the plasma was measured to obtain the blood MYH11 level. Commercially available products of normal plasma samples from 50 individuals (including Caucasians, Hispanics, and Blacks) were purchased and used as negative controls.

The measurement of the MYH11 level in plasma was carried out by sandwich ELISA using a Human Myosin Heavy Chain 11 ELISA Kit (CUSABIO), which uses anti-MYH11 antibodies as an immobilized antibody and as a labeled antibody. HRP was used as the label for the anti-MYH11 antibody, and TMB was used as a substrate for the detection. Coloring was detected using a luminometer. A calibration curve was prepared using standard samples containing smooth muscle myosin at various concentrations. By application to this calibration curve, the MYH11 concentration in each sample was calculated. As the smooth muscle myosin, a recombinant protein included in the above-mentioned kit (CUSABIO) was used.

To identify the region recognized by the anti-MYH11 antibody used in this study, the full length of the MYH11 isoform SM2B (SEQ ID NO:4) was roughly equally divided into two parts to prepare an N-terminal fragment (R1) and a C-terminal fragment (R2), and reactivity of the antibody with each fragment was investigated by ELISA. As a result, the antibody was not reactive with R1, and was reactive only with R2. It was thus confirmed that this antibody recognizes a certain site in the C-terminal half region of MYH11 (in the region of amino acid positions 950 to 1945 of SEQ ID NO:4). Regarding the immobilized antibody in the ELISA kit, it was found to be reactive with the two different fragments of MYH11, and therefore assumed to be a polyclonal antibody.

### <Results>

FIG. 1-1 shows the result of measurement of the blood MYH11 level in 50 normal individuals and a total of 78 patients with arteriosclerosis. Compared to the normal group, the arteriosclerosis patient group clearly showed higher blood MYH11 levels.

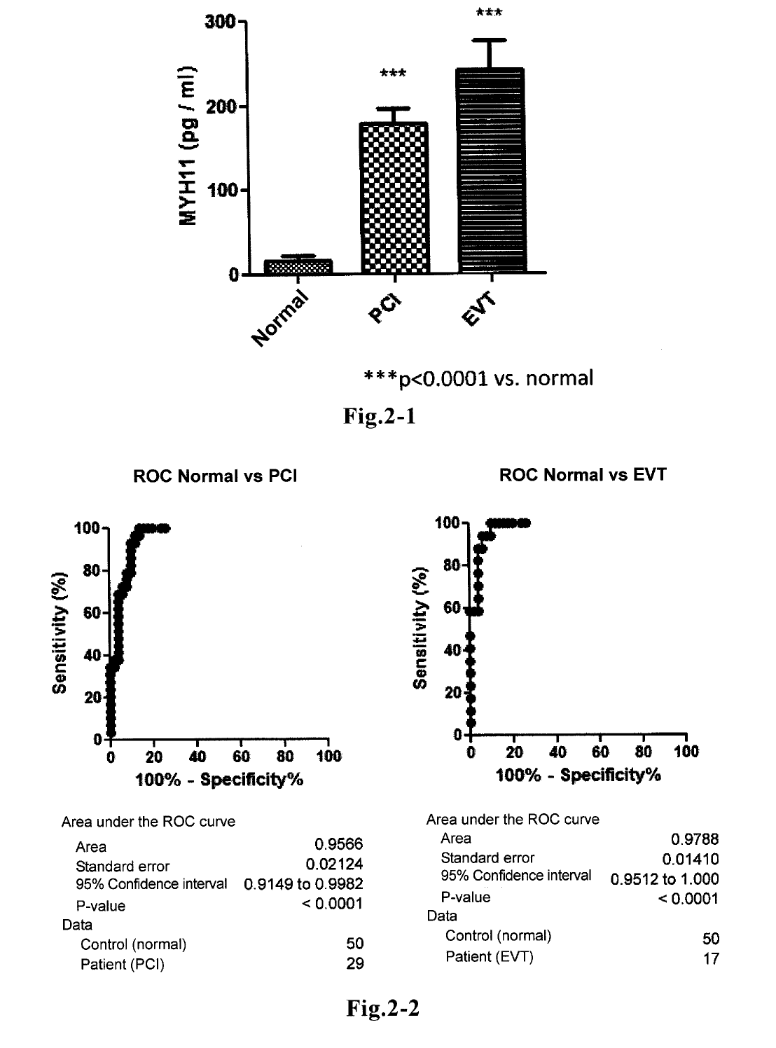

FIG. 1-2 shows the result of evaluation, by ROC analysis, of the capacity of the blood MYH11 level to detect the arteriosclerosis group. This result demonstrated a significantly strong association between the MYH11 concentration in plasma, which is an independent variable, and the outcome, that is, the presence/absence of arteriosclerosis, which is a dichotomous variable. The area under the ROC curve (AUC) was 0.9290. AUC takes a value of from 0.5 to 1.0. An AUC value of from 0.5 to 0.7 is judged as “low accuracy”; an AUC value of from 0.9 to 0.7 is judged as “moderate accuracy”; and an AUC value of from 0.9 to 1.0 is judged as “high accuracy”. In the present case, the AUC value was over 0.9. It was thus shown that diagnosis of arteriosclerotic lesion using the blood MYH11 level as an indicator is highly useful.

The measured values of the blood MYH11 level of 29 patients who received percutaneous coronary intervention (PCI) and 17 patients who received peripheral artery endovascular treatment (EVT) in the group of arteriosclerosis patients were compared with that of the normal group. The result is shown in FIG. 2-1. The patients in these patient groups had highly advanced arteriosclerosis that required therapeutic treatment for a peripheral artery such as a lower limb artery or for a coronary artery. It could be confirmed that the blood MYH11 level is especially high in such cases of very severe arteriosclerotic lesions. FIG. 2-2 shows the result of evaluation, by ROC analysis, of the capacity of the blood MYH11 level to detect each arteriosclerosis patient group. The AUC value was as high as more than 0.95 in both groups.

### 2. Quantification of Myosin Heavy Chain 11 in Plasma by Sandwich ELISA (Part 2)

### <Materials and Methods>

As arteriosclerosis samples, plasma samples derived from 29 patients to which PCI was applied (69.5±1.4 years old; BMI, 22.4±0.5; male:female=26:3) and 17 patients to which EVT was applied (72.4±1.4 years old; BMI, 23.6±1.1; male:female=13:4) out of the arteriosclerosis patient plasma samples in the above-described Section 1 were used. As normal samples, plasma samples from 40 Japanese healthy individuals free of arteriosclerosis and its risk factors (70.4±0.6 years old; BMI, 23.3±0.2; male:female=33:7) were used. Sandwich ELISA was carried out using the same ELISA kit manufactured by CUSABIO as in the Section 1.

### <Results>

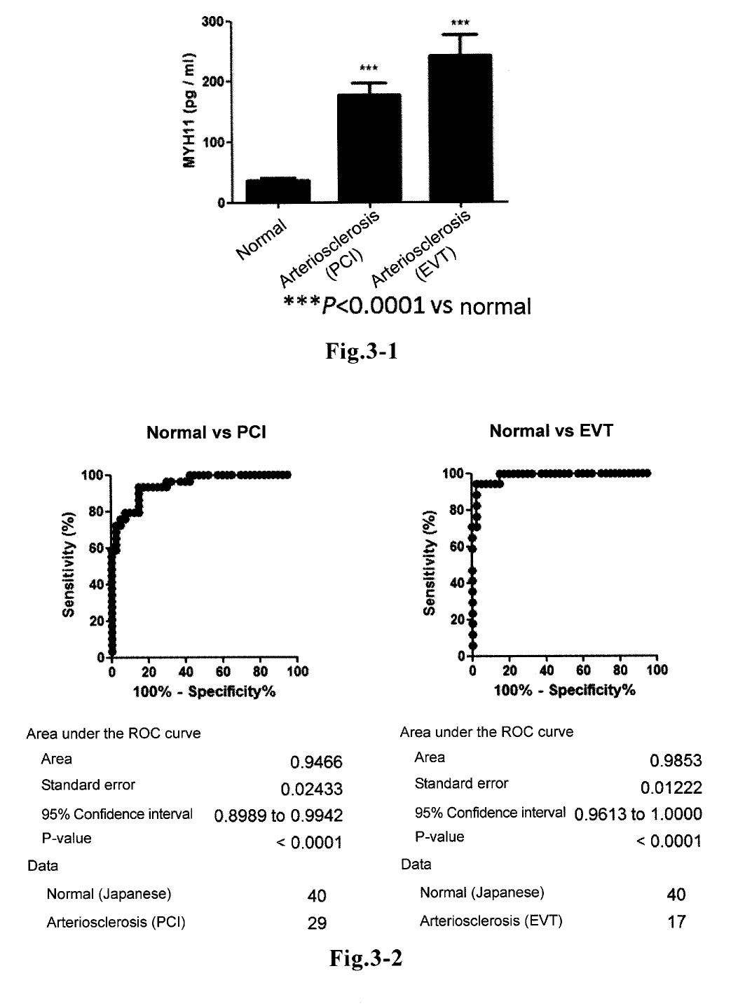

The measured values of the blood MYH11 level of the arteriosclerosis patients to which PCI was applied and the arteriosclerosis patients to which EVT was applied were compared with that of the 40 normal Japanese individuals. The result is shown in FIG. 3-1. FIG. 3-2 shows the result of evaluation, by ROC analysis, of the capacity of the blood MYH11 level to detect each arteriosclerosis patient group. Also in the comparison with the Japanese healthy individuals, the arteriosclerosis patients showed significantly higher blood MYH11 levels, and AUC values were as high as more than 0.9.

### 3. Study of Diagnostic Performance of Blood Myosin Heavy Chain 11 Level for Arteriosclerosis

(1) Comparison of Diagnostic Performance with CRP

C-reactive protein (CRP) is a marker for prediction of cardiovascular events, and it is known that the blood CRP value increases also in cases of arteriosclerosis (Blankengerg S, et al., Circulation 2010; 121: 2388-97). For evaluation of the diagnostic performance of the blood myosin heavy chain 11 level for arteriosclerosis, the plasma CRP level was investigated for the same arteriosclerosis patients (29 PCI patients and 17 EVT patients) and Japanese healthy individuals as in the Section 2.

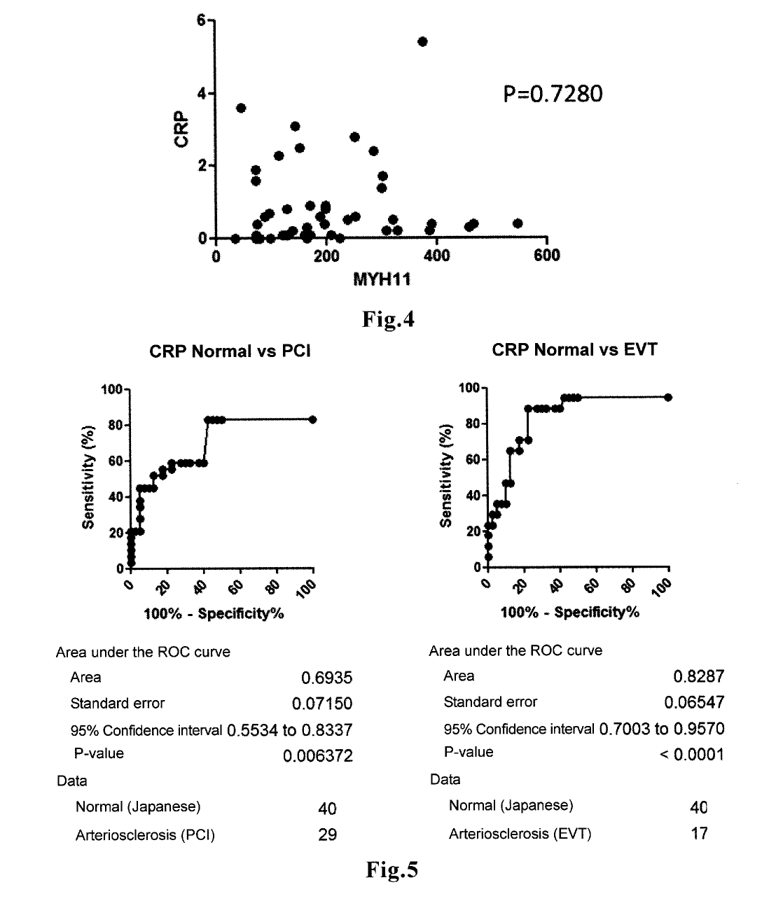

FIG. 4 shows the result of investigation of correlation between the blood MYH11 level and the blood CRP level in the 46 arteriosclerosis patients. It could be confirmed that there is no correlation between them, and that they are factors independent from each other.

FIG. 5 shows the result of evaluation, by ROC analysis, of the capacity of the blood CRP level to detect each arteriosclerosis patient group. The AUC value was as low as less than 0.9 in both groups, and it could thus be confirmed that the diagnostic capacity of CRP for arteriosclerosis is lower than that of MYH11.

(2) Association with Arteriosclerosis Risk Factors

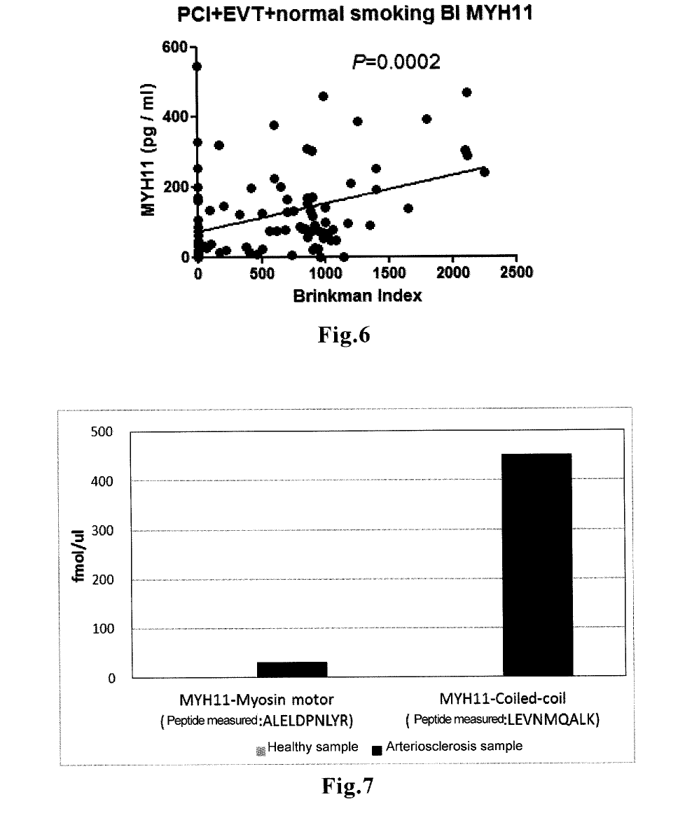

Smoking is one of the major risk factors for arteriosclerosis, and correlation between smoking and arteriosclerosis is known. Correlation between the plasma MYH11 level and the Brinkman index (number of cigarettes smoked per day×number of years of smoking) was investigated in the Japanese healthy individuals, the PCI patients and the EVT patients, and as a result, as shown in FIG. 6, a correlation was found between them. Since slow progression of arteriosclerosis occurs with aging even in healthy individuals, it is thought that the healthy individual population also includes individuals with very mild arteriosclerosis. The PCI patients and the EVT patients are severe cases of arteriosclerosis, which require therapeutic treatment. The data shown in FIG. 6 suggest that even mild arteriosclerosis can be detected based on the blood MYH11 level.

### 4. Analysis of Dynamics of Myosin Heavy Chain 11 in Blood

### <Materials and Methods>

### (1) Preparation of Antibody-Immobilized Microparticles

Magnetic microparticles on which an antibody is to be immobilized (Dynabeads (registered trademark) M-280 Tosylactivated; Thermo Fisher Scientific) were suspended in a buffer described in the protocol, and Anti-MYH11 Human (HPA015310; Cosmo Bio Co., Ltd.) was mixed with the resulting suspension, followed by allowing immobilization of the antibody at 37° C. for 17 hours. After the immobilization of the antibody, the antibody-immobilized microparticles were suspended in 0.05 mol/L Tris-HCl buffer (pH 8.0) supplemented with 10% Blockmaster CE510 (JSR), and the resulting suspension was incubated at 37° C. for 5 hours to perform blocking, thereby obtaining anti-MYH11 antibody-immobilized magnetic microparticles (A) at 0.2 mg/mL beads.

### (2) Preparation of Internal Standard Peptide

A peptide derived from the myosin motor structure (as a peptide derived from the N-terminal structure; ALELDPNLYR; the region of residue positions 760 to 769 of SEQ ID NO:4) and a peptide derived from the coiled-coil structure (as a peptide derived from the C-terminal structure; LEVNMQALK; the region of residue positions 1572 to 1580 of SEQ ID NO:4) were selected as target peptides, and the peptides labeled with stable isotopes (¹³C and ¹⁵N) were synthesized (AQUA peptide; Sigma-Aldrich).

### (3) Preparation of Measurement Sample

Among the patient samples used in the Section 1, plasma samples of several patients who showed relatively high values of the plasma MYH11 level were pooled to provide a sample to be used as a patient sample. Plasma samples from six Japanese healthy individuals were pooled to provide a healthy individual sample to be used as a negative control.

For immune reaction using the magnetic microparticles, (A) was suspended in a solution prepared by dilution of the sample with PBST, and the reaction was allowed to proceed with stirring for 3 hours at room temperature. Using 0.1 N hydrochloric acid supplemented with 8 M urea as an eluent, an eluate (B) was obtained. (B) was neutralized with 1 M Tris, and then subjected to reductive alkylation according to a conventional method. Trypsin was added thereto to a final concentration of 5.0 μg/mL, and the resulting mixture was incubated at 37° C. for 17 hours to obtain a protein digestion product (C). To the resulting solution of (C), the internal standard peptides (10 fmol) were added, and the resulting mixture was subjected to desalting/concentration with GL-Tip SDB (GL Science) to obtain an MRM measurement sample (D).

### (4) Obtaining Peptide Information by Multiple Reaction Monitoring (MRM) Method

The MRM measurement sample (D) obtained was subjected to a triple quadrupole-type mass spectrometer QTRAP 5500 (AB SCIEX) to perform measurement of the two peptides, MYH11-myosin motor peptide and MYH11-coiled-coil peptide. The MRM methods for detection and quantification of these peptides are shown in Table 1. The measurement results were analyzed with Skyline software. Signal reliability was evaluated by confirming that the elution time of the MRM signal in the quantitation channel (Quantitation) accorded with that of the corresponding signal in the verification channel (Verification). Subsequently, in the quantitation channel for each peptide, the peak area ratio of the Light peptide to the Heavy peptide was calculated to calculate the molar concentrations of these peptides contained in the analysis sample.

TABLE 1

| | | | | MRM Transitions | | | |
|---|---|---|---|---|---|---|---|
| Peptide Name | Position | Sequence | Label | Q1 | CE (V) | Q3 | Analysis |
| MYH11-Myosin Motor | 760-769 | ALELDPNLYR | Light | 602.3 (2+) | 30.5 | 890.5 (y7+) | Verification |
| | | | | | | 777.4 (y6+) | Quantitation |
| | | | Arg10: Heavy (+10) | 607.3 (2+) | 30.5 | 900.5 (y7+) | Verification |
| | | | | | | 787.4 (y6+) | Quantitation |
| MYH11-Coiled-Coil | 1572-1580 | LEVNMQALK | Light | 523.3 (2+) | 27.7 | 803.4 (y7+) | Verification |
| | | | | | | 704.4 (y6+) | Quantitation |
| | | | Lys9: Heavy (+6) | 526.3 (2+) | 27.7 | 809.5 (y7+) | Verification |
| | | | | | | 710.4 (y6+) | Quantitation |

### <Results>

Using the internal standard peptides as indices, the molar concentration of MYH11 present in the sample was measured from the quantitative channels for the two target peptides. The results are shown in FIG. 7. In the healthy individual sample used for comparison, the concentrations of both peptides were below the detection limit. On the other hand, in the arteriosclerosis sample, the peptide derived from the C-terminal structure of MYH11 was detected at a very high level, and this level was found to be significantly higher than in the healthy individuals. Regarding the peptide derived from the N-terminal structure, it was revealed that, similarly to the peptide derived from the C-terminal structure, the peptide derived from the N-terminal structure was detected in the arteriosclerosis sample at a significantly higher level than in the healthy individuals, although its level was lower than that of the peptide derived from the C-terminal structure.

5. Association of Blood MYH11 Level with Prognosis of Arteriosclerotic Disease

The postoperative course of patients who received PCI or EVT was monitored (monitoring period, 1 to 2.5 years). As a result, three cases of death were found. Since these cases of death did not occur immediately after the intervention, it is apparent that they were not caused by the intervention procedure.

1. Died of infection 64 days after EVT intervention

2. Died of infection 294 days after EVT intervention

3. Died of disseminated intravascular coagulation (DIC) 163 days after PCI intervention

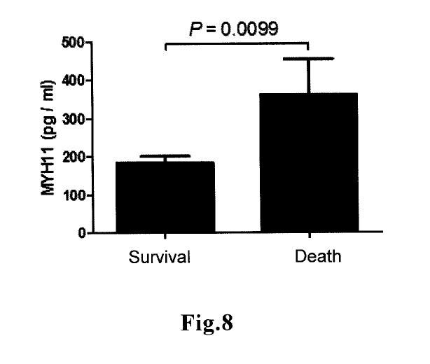

The blood MYH11 level before the intervention, immediately after the intervention, or within several days after the intervention was compared between the death group and the surviving patient group. As a result, as shown in FIG. 8, the blood MYH11 level was higher in the death group. It is known that the risk of severe infection is elevated in peripheral arterial diseases caused by stenosis or occlusion of a peripheral artery (https://www.nhIbi.nih.gov/health/health-topics/topics/atherosclerosis). Further, DIC is a disease state that may occur due to various underlying diseases, and coronary stenosis requiring intervention is one of the underlying diseases of DIC. This data suggests that the blood MYH11 level is useful also as an indicator of prognosis of arteriosclerotic diseases.

1. A method for detecting arteriosclerosis, comprising measuring the myosin heavy chain 11 level in a blood sample isolated from a subject, wherein a myosin heavy chain 11 level higher than that in a healthy individual is indicative of the presence of arteriosclerosis in the subject.

2. An arteriosclerosis detection kit comprising an anti-myosin heavy chain 11 antibody or an antigen-binding fragment thereof.


SEQUENCE LISTING (4 sequences)

SEQ ID NO: 1 - protein, 1972 aa, Homo sapiens
MAQKGQLSDDEKFLFVDKNFINSPVAQADWAAKRLVWVPSEKQGFEAASIKEEKGDEVVVELVENGKKVTVGKDDIQKMNPPKFSKVEDMAELTCLNEASVLHNLRERYFSGLIYTYSGLFCVVVNPYKHLPIYSEKIVDMYKGKKRHEMPPHIYAIADTAYRSMLQDREDQSILCTGESGAGKTENTKKVIQYLAVVASSHKGKKDTSITGELEKQLLQANPILEAFGNAKTVKNDNSSRFGKFIRINFDVTGYIVGANIETYLLEKSRAIRQARDERTFHIFYYMIAGAKEKMRSDLLLEGFNNYTFLSNGFVPIPAAQDDEMFQETVEAMAIMGFSEEEQLSILKVVSSVLQLGNIVFKKERNTDQASMPDNTAAQKVCHLMGINVTDFTRSILTPRIKVGRDVVQKAQTKEQADFAVEALAKATYERLFRWILTRVNKALDKTHRQGASFLGILDIAGFEIFEVNSFEQLCINYTNEKLQQLFNHTMFILEQEEYQREGIEWNFIDFGLDLQPCIELIERPNNPPGVLALLDEECWFPKATDKSFVEKLCTEQGSHPKFQKPKQLKDKTEFSIIHYAGKVDYNASAWLTKNMDPLNDNVTSLLNASSDKFVADLWKDVDRIVGLDQMAKMTESSLPSASKTKKGMFRTVGQLYKEQLGKLMTTLRNTTPNFVRCIIPNHEKRSGKLDAFLVLEQLRCNGVLEGIRICRQGFPNRIVFQEFRQRYEILAANAIPKGFMDGKQACILMIKALELDPNLYRIGQSKIFFRTGVLAHLEEERDLKITDVIMAFQAMCRGYLARKAFAKRQQQLTAMKVIQRNCAAYLKLRNWQWWRLFTKVKPLLQVTRQEEEMQAKEDELQKTKERQQKAENELKELEQKHSQLTEEKNLLQEQLQAETELYAEAEEMRVRLAAKKQELEEILHEMEARLEEEEDRGQQLQAERKKMAQQMLDLEEQLEEEEAARQKLQLEKVTAEAKIKKLEDEILVMDDQNNKLSKERKLLEERISDLTTNLAEEEEKAKNLTKLKNKHESMISELEVRLKKEEKSRQELEKLKRKLEGDASDFHEQIADLQAQIAELKMQLAKKEEELQAALARLDDEIAQKNNALKKIRELEGHISDLQEDLDSERAARNKAEKQKRDLGEELEALKTELEDTLDSTATQQELRAKREQEVTVLKKALDEETRSHEAQVQEMRQKHAQAVEELTEQLEQFKRAKANLDKNKQTLEKENADLAGELRVLGQAKQEVEHKKKKLEAQVQELQSKCSDGERARAELNDKVHKLQNEVESVTGMLNEAEGKAIKLAKDVASLSSQLQDTQELLQEETRQKLNVSTKLRQLEEERNSLQDQLDEEMEAKQNLERHISTLNIQLSDSKKKLQDFASTVEALEEGKKRFQKEIENLTQQYEEKAAAYDKLEKTKNRLQQELDDLVVDLDNQRQLVSNLEKKQRKFDQLLAEEKNISSKYADERDRAEAEAREKETKALSLARALEEALEAKEELERTNKMLKAEMEDLVSSKDDVGKNVHELEKSKRALETQMEEMKTQLEELEDELQATEDAKLRLEVNMQALKGQFERDLQARDEQNEEKRRQLQRQLHEYETELEDERKQRALAAAAKKKLEGDLKDLELQADSAIKGREEAIKQLRKLQAQMKDFQRELEDARASRDEIFATAKENEKKAKSLEADLMQLQEDLAAAERARKQADLEKEELAEELASSLSGRNALQDEKRRLEARIAQLEEELEEEQGNMEAMSDRVRKATQQAEQLSNELATERSTAQKNESARQQLERQNKELRSKLHEMEGAVKSKFKSTIAALEAKIAQLEEQVEQEAREKQAATKSLKQKDKKLKEILLQVEDERKMAEQYKEQAEKGNARVKQLKRQLEEAEEESQRINANRRKLQRELDEATESNEAMGREVNALKSKLRRGNETSFVPSRRSGGRRVIENADGSEEETDTRDADFNGTKASE

SEQ ID NO: 2 - protein, 1979 aa, Homo sapiens
MAQKGQLSDDEKFLFVDKNFINSPVAQADWAAKRLVWVPSEKQGFEAASIKEEKGDEVVVELVENGKKVTVGKDDIQKMNPPKFSKVEDMAELTCLNEASVLHNLRERYFSGLIYTYSGLFCVVVNPYKHLPIYSEKIVDMYKGKKRHEMPPHIYAIADTAYRSMLQDREDQSILCTGESGAGKTENTKKVIQYLAVVASSHKGKKDTSITQGPSFAYGELEKQLLQANPILEAFGNAKTVKNDNSSRFGKFIRINFDVTGYIVGANIETYLLEKSRAIRQARDERTFHIFYYMIAGAKEKMRSDLLLEGFNNYTFLSNGFVPIPAAQDDEMFQETVEAMAIMGFSEEEQLSILKVVSSVLQLGNIVFKKERNTDQASMPDNTAAQKVCHLMGINVTDFTRSILTPRIKVGRDVVQKAQTKEQADFAVEALAKATYERLFRWILTRVNKALDKTHRQGASFLGILDIAGFEIFEVNSFEQLCINYTNEKLQQLFNHTMFILEQEEYQREGIEWNFIDFGLDLQPCIELIERPNNPPGVLALLDEECWFPKATDKSFVEKLCTEQGSHPKFQKPKQLKDKTEFSIIHYAGKVDYNASAWLTKNMDPLNDNVTSLLNASSDKFVADLWKDVDRIVGLDQMAKMTESSLPSASKTKKGMFRTVGQLYKEQLGKLMTTLRNTTPNFVRCIIPNHEKRSGKLDAFLVLEQLRCNGVLEGIRICRQGFPNRIVFQEFRQRYEILAANAIPKGFMDGKQACILMIKALELDPNLYRIGQSKIFFRTGVLAHLEEERDLKITDVIMAFQAMCRGYLARKAFAKRQQQLTAMKVIQRNCAAYLKLRNWQWWRLFTKVKPLLQVTRQEEEMQAKEDELQKTKERQQKAENELKELEQKHSQLTEEKNLLQEQLQAETELYAEAEEMRVRLAAKKQELEEILHEMEARLEEEEDRGQQLQAERKKMAQQMLDLEEQLEEEEAARQKLQLEKVTAEAKIKKLEDEILVMDDQNNKLSKERKLLEERISDLTTNLAEEEEKAKNLTKLKNKHESMISELEVRLKKEEKSRQELEKLKRKLEGDASDFHEQIADLQAQIAELKMQLAKKEEELQAALARLDDEIAQKNNALKKIRELEGHISDLQEDLDSERAARNKAEKQKRDLGEELEALKTELEDTLDSTATQQELRAKREQEVTVLKKALDEETRSHEAQVQEMRQKHAQAVEELTEQLEQFKRAKANLDKNKQTLEKENADLAGELRVLGQAKQEVEHKKKKLEAQVQELQSKCSDGERARAELNDKVHKLQNEVESVTGMLNEAEGKAIKLAKDVASLSSQLQDTQELLQEETRQKLNVSTKLRQLEEERNSLQDQLDEEMEAKQNLERHISTLNIQLSDSKKKLQDFASTVEALEEGKKRFQKEIENLTQQYEEKAAAYDKLEKTKNRLQQELDDLVVDLDNQRQLVSNLEKKQRKFDQLLAEEKNISSKYADERDRAEAEAREKETKALSLARALEEALEAKEELERTNKMLKAEMEDLVSSKDDVGKNVHELEKSKRALETQMEEMKTQLEELEDELQATEDAKLRLEVNMQALKGQFERDLQARDEQNEEKRRQLQRQLHEYETELEDERKQRALAAAAKKKLEGDLKDLELQADSAIKGREEAIKQLRKLQAQMKDFQRELEDARASRDEIFATAKENEKKAKSLEADLMQLQEDLAAAERARKQADLEKEELAEELASSLSGRNALQDEKRRLEARIAQLEEELEEEQGNMEAMSDRVRKATQQAEQLSNELATERSTAQKNESARQQLERQNKELRSKLHEMEGAVKSKFKSTIAALEAKIAQLEEQVEQEAREKQAATKSLKQKDKKLKEILLQVEDERKMAEQYKEQAEKGNARVKQLKRQLEEAEEESQRINANRRKLQRELDEATESNEAMGREVNALKSKLRRGNETSFVPSRRSGGRRVIENADGSEEETDTRDADFNGTKASE

SEQ ID NO: 3 - protein, 1938 aa, Homo sapiens
MAQKGQLSDDEKFLFVDKNFINSPVAQADWAAKRLVWVPSEKQGFEAASIKEEKGDEVVVELVENGKKVTVGKDDIQKMNPPKFSKVEDMAELTCLNEASVLHNLRERYFSGLIYTYSGLFCVVVNPYKHLPIYSEKIVDMYKGKKRHEMPPHIYAIADTAYRSMLQDREDQSILCTGESGAGKTENTKKVIQYLAVVASSHKGKKDTSITGELEKQLLQANPILEAFGNAKTVKNDNSSRFGKFIRINFDVTGYIVGANIETYLLEKSRAIRQARDERTFHIFYYMIAGAKEKMRSDLLLEGFNNYTFLSNGFVPIPAAQDDEMFQETVEAMAIMGFSEEEQLSILKVVSSVLQLGNIVFKKERNTDQASMPDNTAAQKVCHLMGINVTDFTRSILTPRIKVGRDVVQKAQTKEQADFAVEALAKATYERLFRWILTRVNKALDKTHRQGASFLGILDIAGFEIFEVNSFEQLCINYTNEKLQQLFNHTMFILEQEEYQREGIEWNFIDFGLDLQPCIELIERPNNPPGVLALLDEECWFPKATDKSFVEKLCTEQGSHPKFQKPKQLKDKTEFSIIHYAGKVDYNASAWLTKNMDPLNDNVTSLLNASSDKFVADLWKDVDRIVGLDQMAKMTESSLPSASKTKKGMFRTVGQLYKEQLGKLMTTLRNTTPNFVRCIIPNHEKRSGKLDAFLVLEQLRCNGVLEGIRICRQGFPNRIVFQEFRQRYEILAANAIPKGFMDGKQACILMIKALELDPNLYRIGQSKIFFRTGVLAHLEEERDLKITDVIMAFQAMCRGYLARKAFAKRQQQLTAMKVIQRNCAAYLKLRNWQWWRLFTKVKPLLQVTRQEEEMQAKEDELQKTKERQQKAENELKELEQKHSQLTEEKNLLQEQLQAETELYAEAEEMRVRLAAKKQELEEILHEMEARLEEEEDRGQQLQAERKKMAQQMLDLEEQLEEEEAARQKLQLEKVTAEAKIKKLEDEILVMDDQNNKLSKERKLLEERISDLTTNLAEEEEKAKNLTKLKNKHESMISELEVRLKKEEKSRQELEKLKRKLEGDASDFHEQIADLQAQIAELKMQLAKKEEELQAALARLDDEIAQKNNALKKIRELEGHISDLQEDLDSERAARNKAEKQKRDLGEELEALKTELEDTLDSTATQQELRAKREQEVTVLKKALDEETRSHEAQVQEMRQKHAQAVEELTEQLEQFKRAKANLDKNKQTLEKENADLAGELRVLGQAKQEVEHKKKKLEAQVQELQSKCSDGERARAELNDKVHKLQNEVESVTGMLNEAEGKAIKLAKDVASLSSQLQDTQELLQEETRQKLNVSTKLRQLEEERNSLQDQLDEEMEAKQNLERHISTLNIQLSDSKKKLQDFASTVEALEEGKKRFQKEIENLTQQYEEKAAAYDKLEKTKNRLQQELDDLVVDLDNQRQLVSNLEKKQRKFDQLLAEEKNISSKYADERDRAEAEAREKETKALSLARALEEALEAKEELERTNKMLKAEMEDLVSSKDDVGKNVHELEKSKRALETQMEEMKTQLEELEDELQATEDAKLRLEVNMQALKGQFERDLQARDEQNEEKRRQLQRQLHEYETELEDERKQRALAAAAKKKLEGDLKDLELQADSAIKGREEAIKQLRKLQAQMKDFQRELEDARASRDEIFATAKENEKKAKSLEADLMQLQEDLAAAERARKQADLEKEELAEELASSLSGRNALQDEKRRLEARIAQLEEELEEEQGNMEAMSDRVRKATQQAEQLSNELATERSTAQKNESARQQLERQNKELRSKLHEMEGAVKSKFKSTIAALEAKIAQLEEQVEQEAREKQAATKSLKQKDKKLKEILLQVEDERKMAEQYKEQAEKGNARVKQLKRQLEEAEEESQRINANRRKLQRELDEATESNEAMGREVNALKSKLRGPPPQETSQ

SEQ ID NO: 4 - protein, 1945 aa, Homo sapiens
MAQKGQLSDDEKFLFVDKNFINSPVAQADWAAKRLVWVPSEKQGFEAASIKEEKGDEVVVELVENGKKVTVGKDDIQKMNPPKFSKVEDMAELTCLNEASVLHNLRERYFSGLIYTYSGLFCVVVNPYKHLPIYSEKIVDMYKGKKRHEMPPHIYAIADTAYRSMLQDREDQSILCTGESGAGKTENTKKVIQYLAVVASSHKGKKDTSITQGPSFAYGELEKQLLQANPILEAFGNAKTVKNDNSSRFGKFIRINFDVTGYIVGANIETYLLEKSRAIRQARDERTFHIFYYMIAGAKEKMRSDLLLEGFNNYTFLSNGFVPIPAAQDDEMFQETVEAMAIMGFSEEEQLSILKVVSSVLQLGNIVFKKERNTDQASMPDNTAAQKVCHLMGINVTDFTRSILTPRIKVGRDVVQKAQTKEQADFAVEALAKATYERLFRWILTRVNKALDKTHRQGASFLGILDIAGFEIFEVNSFEQLCINYTNEKLQQLFNHTMFILEQEEYQREGIEWNFIDFGLDLQPCIELIERPNNPPGVLALLDEECWFPKATDKSFVEKLCTEQGSHPKFQKPKQLKDKTEFSIIHYAGKVDYNASAWLTKNMDPLNDNVTSLLNASSDKFVADLWKDVDRIVGLDQMAKMTESSLPSASKTKKGMFRTVGQLYKEQLGKLMTTLRNTTPNFVRCIIPNHEKRSGKLDAFLVLEQLRCNGVLEGIRICRQGFPNRIVFQEFRQRYEILAANAIPKGFMDGKQACILMIKALELDPNLYRIGQSKIFFRTGVLAHLEEERDLKITDVIMAFQAMCRGYLARKAFAKRQQQLTAMKVIQRNCAAYLKLRNWQWWRLFTKVKPLLQVTRQEEEMQAKEDELQKTKERQQKAENELKELEQKHSQLTEEKNLLQEQLQAETELYAEAEEMRVRLAAKKQELEEILHEMEARLEEEEDRGQQLQAERKKMAQQMLDLEEQLEEEEAARQKLQLEKVTAEAKIKKLEDEILVMDDQNNKLSKERKLLEERISDLTTNLAEEEEKAKNLTKLKNKHESMISELEVRLKKEEKSRQELEKLKRKLEGDASDFHEQIADLQAQIAELKMQLAKKEEELQAALARLDDEIAQKNNALKKIRELEGHISDLQEDLDSERAARNKAEKQKRDLGEELEALKTELEDTLDSTATQQELRAKREQEVTVLKKALDEETRSHEAQVQEMRQKHAQAVEELTEQLEQFKRAKANLDKNKQTLEKENADLAGELRVLGQAKQEVEHKKKKLEAQVQELQSKCSDGERARAELNDKVHKLQNEVESVTGMLNEAEGKAIKLAKDVASLSSQLQDTQELLQEETRQKLNVSTKLRQLEEERNSLQDQLDEEMEAKQNLERHISTLNIQLSDSKKKLQDFASTVEALEEGKKRFQKEIENLTQQYEEKAAAYDKLEKTKNRLQQELDDLVVDLDNQRQLVSNLEKKQRKFDQLLAEEKNISSKYADERDRAEAEAREKETKALSLARALEEALEAKEELERTNKMLKAEMEDLVSSKDDVGKNVHELEKSKRALETQMEEMKTQLEELEDELQATEDAKLRLEVNMQALKGQFERDLQARDEQNEEKRRQLQRQLHEYETELEDERKQRALAAAAKKKLEGDLKDLELQADSAIKGREEAIKQLRKLQAQMKDFQRELEDARASRDEIFATAKENEKKAKSLEADLMQLQEDLAAAERARKQADLEKEELAEELASSLSGRNALQDEKRRLEARIAQLEEELEEEQGNMEAMSDRVRKATQQAEQLSNELATERSTAQKNESARQQLERQNKELRSKLHEMEGAVKSKFKSTIAALEAKIAQLEEQVEQEAREKQAATKSLKQKDKKLKEILLQVEDERKMAEQYKEQAEKGNARVKQLKRQLEEAEEESQRINANRRKLQRELDEATESNEAMGREVNALKSKLRGPPPQETSQ